(19) United States
(12) Patent Application Publication
(10) Pub. No.: US 2006/0252670 A1
(43) Pub. Date: Nov. 9, 2006

(54) Method of reducing drug-induced adverse side effects in a patient

(75) Inventors: Stefano Fiorucci, Perugia (IT); Roberto Pellicciari, Perugia (IT); Mark Pruzanski, New York, NY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Intercept Pharmaceuticals Inc., New York, NY (US)

(21) Appl. No.: 11/250,298

(22) Filed: Oct. 13, 2005

Related U.S. Application Data
(60) Provisional application No. 60/619,381, filed on Oct. 14, 2004.

(51) Int. Cl. A61K 38/28; A61K 31/426; A61K 31/17; A61K 31/155; A61K 31/175

(52) U.S. Cl. 514/3; 514/369; 514/594; 514/592; 514/635

(57) ABSTRACT

The invention relates to the discovery that farnesoid X receptor (FXR) agonists can be used in combination with peroxisome proliferation activated receptor gamma (PPARγ) agonists to reduce drug-induced adverse side effects in patients suffering from conditions such as insulin resistance, Type II diabetes, metabolic syndrome, non-alcoholic fatty liver disease (NAFLD), non-alcoholic steatohepatitis (NASH), and heart disease. Particularly, the present invention encompasses methods for treating patients suffering from drug-induced adverse side effects with selective PPARγ, dual PPARα/γ and pan PPARα/γ/δ agonists in combination with FXR agonists.

### CROSS-REFERENCES TO RELATED APPLICATIONS

This application claims the benefit of U.S. Provisional Application No. 60/619,381, filed Oct. 14, 2004, the full disclosure of which is incorporated herein by reference.

### FIELD OF THE INVENTION

The invention relates to the discovery that gamma peroxisome proliferation activated receptor (PPARγ) agonists can be used in combination with farnesoid X receptor (FXR) agonists to reduce drug-induced adverse side effects in patients suffering from conditions such as insulin resistance, Type II diabetes and heart disease. Particularly, the present invention encompasses methods for treating patients suffering from drug-induced adverse side effects with selective PPARγ-, dual PPARα/γ- and pan PPARα/γ/δ agonists in combination with FXR agonists.

### BACKGROUND OF THE INVENTION

PPARγ agonists are therapeutics for Type II diabetes, insulin resistance and for a variety of metabolic and cardiovascular diseases. There is a relationship between diabetes and cardiovascular disease (CVD). CVD is the main cause of death in diabetic patients. Type II diabetes, also referred to as adult-onset diabetes or non-insulin-dependent diabetes, is a condition where insulin is produced but the body cannot effectively use it.

A pre-diabetic state termed insulin resistance is a condition that increases the likelihood of developing Type II diabetes and heart disease (HD). In a person who suffers from insulin resistance, muscle, fat, and liver cells do not metabolize insulin properly. The pancreas tries to keep up with the demand for insulin by producing more. Eventually, the pancreas cannot keep up with the body's need for insulin, and excess glucose builds up in the bloodstream. Usually, people with insulin resistance have high levels of blood glucose and high levels of insulin circulating in their blood simultaneously. Their high fasting blood glucose levels normally range around 110 mg/dL or higher. They usually also have excess weight around the waist, high LDL blood cholesterol levels, low HDL cholesterol levels (e.g., below 40 mg/dL for men and below 50 mg/dL for women), high levels of triglycerides (e.g., 150 mg/dL or higher), and high blood pressure (e.g., 130/85 mmHg or higher), all conditions that also put the heart at risk. This combination of symptoms is referred to as the metabolic syndrome, or the insulin resistance syndrome (formerly called Syndrome X).

Individuals with blood glucose levels that are higher than normal but not yet in the diabetic range are considered to have “pre-diabetes”. This condition is also referred to as impaired fasting glucose (IFG) or impaired glucose tolerance (IGT), depending on the test used to diagnose it. In a cross-section of U.S. adults aged 40 to 74, tested during the period 1988 to 1994, 33.8 percent had IFG, 15.4 percent had IGT, and 40.1 percent had pre-diabetes (IGT or IFG or both). The numbers of U.S. individuals afflicted with IGT and/or IGF are believed to increase as time passes. Persons who suffer from pre-diabetes have a higher risk of developing Type II diabetes. Studies have shown that most individuals who are diagnosed with pre-diabetes eventually develop Type II diabetes within 10 years (i.e., unless they lose 5 to 7 percent of their body weight). Such individuals also have a higher risk of developing heart disease.

Insulin resistance and pre-diabetes often have no symptoms. Afflicted individuals can have one or both conditions for several years without noticing anything. An increasingly recognized condition in obese individuals with pre-diabetic insulin resistance or Type II diabetes is non-alcoholic fatty liver disease (NAFLD) or non-alcoholic steatohepatitis (NASH). Individuals with NAFLD or NASH are at significant risk of developing progressive liver fibrosis that may lead to cirrhosis and liver failure.

Individuals with a severe form of insulin resistance may get dark patches of skin, usually on the back of the neck (e.g., dark ring around the neck). Other possible sites for these dark patches include elbows, knees, knuckles, and armpits. This condition is called acanthosis nigricans (Insulin Resistance and Pre-Diabetes, NIH Publication No. 04-4893 (May 2004).

During the past decade, PPAR agonists have been introduced for the treatment of Type II diabetes. These drugs are used in monotherapy or in combination therapy and have been shown to be effective in lowering blood glucose. Among the PPARγ agonists are thiazolidinediones (TZDs). Examples include rosiglitazone, pioglitazone, and troglitazone.

There have been adverse side effects with PPARγ agonists. Troglitazone, the first agent of this class of drugs to be approved, was effective in controlling glycemia but was removed from the market because of serious liver toxicity. Rosiglitazone and pioglitazone are indicated either as monotherapy or in combination with sulfonylurea, metformin, or insulin when diet, exercise and a single agent do not result in adequate glycemic control. In addition to lowering blood glucose, both drugs may benefit cardiovascular parameters, such as lipids, blood pressure, inflammatory biomarkers, endothelial function, and fibrinolytic status. As such, the beneficial effects of TZDs on glycemia and cardiovascular risk factors have made these drugs effective agents in patients with Type II diabetes who are at high risk for cardiovascular disease (CVD) (Nesto et al. (January 2004) Diabetic Care 27(1):256-263). There is also evidence that PPARγ agonists may possess antifibrotic and anti-inflammatory effects in liver disease associated with obesity, insulin resistance and Type II diabetes, including non-alcoholic fatty liver disease (NAFLD) and non-alcoholic steatohepatitis (NASH). However, many patients are unable to benefit from these new drugs because of their adverse dose dependent side effects which include weight gain and excessive fluid retention or edema.

Because of the PPARγ agonist side effects, there is value in potentiating the PPARγ agonists to lower their effective doses and to simultaneously avoid or reduce the adverse side effects. This invention provides one solution for this.

### SUMMARY OF THE INVENTION

The present invention is directed to a method for treating patients suffering from a condition such as insulin resistance, Type II diabetes, metabolic syndrome, non-alcoholic fatty liver disease (NAFLD), non-alcoholic steatohepatitis (NASH) and/or heart disease with a combination of PPARγ agonists and FXR agonists. Particularly, selective PPARγ, dual PPARα/γ and/or pan PPARα/γ/δ agonists in combination with FXR agonists are contemplated by the present invention. The instant invention offers a multitude of advantages over conventional treatments. One particular advantage is the reduction of adverse side effects that are generally induced by the use of various PPARγ agonists. Side effects like edema or weight gain are frequent side effects in patients who are treated with PPARγ agonists. An advantage of the present invention is that the FXR agonist potentiates the therapeutic effect of selective PPARγ, dual PPARα/γ and pan PPARα/γ/δ agonists. As such, the patient can be administered a lower dose of a PPARγ agonist in combination with an FXR agonist and still achieve the same beneficial therapeutic effect normally associated with a higher dose of the same PPARγ agonist. Therefore, the treatment of patients with PPARγ agonists in combination with FXR agonists alleviates PPARγ associated side effects like edema or weight gain considerably.

Yet another advantage of the present invention is the use of selective PPARγ, dual PPARα/γ and/or pan PPARα/γ/δ agonists in combination with FXR agonists to treat patients who suffer from advanced stages of heart disease. These patients are generally intolerant of conventional treatment with PPARγ agonists. However, the combination treatment of the instant invention achieves a beneficial therapeutic effect as well as a reduction of adverse side effects in patients suffering from heart disease.

More specifically, the present invention provides a method of reducing adverse side effects in a human subject suffering from side effects induced by a PPARγ agonist. The method comprises coadministering to the human subject an FXR agonist in an amount sufficient to potentiate an insulin sensitizing effect of the PPARγ agonist, thereby reducing the amount of the PPARγ agonist taken by the human subject such that the side effects are lessened while the insulin sensitizing effect is preserved. The insulin sensitizing effect of the PPARγ agonist remains as potent at a lower dose as compared to a higher dose as a result of coadministration of the FXR agonist to the human subject. The side effects include, but are not limited to, edema and weight gain. More specifically, the human subject may be a patient suffering from pre-diabetic insulin resistance, metabolic syndrome, Type II diabetes, non-alcoholic fatty liver disease (NAFLD), non-alcoholic steatohepatitis (NASH) and/or heart disease. Preferably, the PPARγ agonist is selected from a group of selective PPARγ agonists or modulators, dual PPARγ/α agonists and/or pan PPARγ/α/δ agonists.

In addition, it is described herein that the above method is preferred wherein said FXR agonist achieves a 25% potentiation of the insulin sensitizing effect of the PPARγ agonist. This can be demonstrated by standard measures of glycemic control The human subject is typically a patient suffering from pre-diabetic insulin resistance or Type II diabetes. The patient optionally may further suffer from a disease, condition, sign or symptom selected from the group consisting of insulin resistance, obesity, non-alcoholic fatty liver disease (NAFLD) or non-alcoholic steatohepatitis (NASH), hyperlipidemia, dyslipidemia, hypercholesterolemia, hypertriglyceridemia, high fasting blood glucose, fluid retention, edema, retinopathy, kidney disease, peripheral neuropathy, hypertension, atherosclerosis and heart failure.

In addition, the above method of claim is applicable wherein said human subject is a patient suffering from symptoms selected from the group consisting of hypertension, atherosclerosis, peripheral vascular disease, and congestive heart failure.

Preferred PPARγ agonist is selected from the group consisting of rosiglitazone and pioglitazone. The invention finds use where the rosiglitazone is administered to a patient in an amount from about 0.5 mg to about 10 mg po qd and where pioglitazone is administered to a patient in an amount from about 3 mg to about 50 mg po qd.

The above methods further provide for use of FXR agonists where said PPARγ agonist is selected from the group consisting of dual PPARγ/α agonists and pan PPARγ/α/δ agonists. These include but are not limited to the group consisting of muraglitazar, galida tesaglitazar, naveglitazar (LY818) and LY929. Pan PPARγ/α/δ agonist are selected from the group consisting of GSK's 677954 and PLX204.

For practicing the above methods the amount of the FXR agonist is preferably administered between about 0.1 mg/kg qd and about 10 mg/kg qd.

Alternatively, the above method is useful where the human subject is a patient suffering from heart disease, and is intolerant of being treated with a PPARγ agonist alone or in combination with a second agent selected from the group consisting of metformin, sulfonylurea and insulin.

The preferred PPAR agonists are PPARγ agonists selected from the group consisting of azelaoyl PAF, 2-bromohexadecanoic acid, ciglitizone, clofibrate, 1 5-deoxy-12,14 prostaglandin J2, fenofibrate, Fmoc-Leu-OH, GW1929, GW7647, 8(S)-hydroxy-(5Z,9E,1Z,14Z)-eicosatetraenoic acid (8(S)-HETE), leukotriene B4, LY- 171,883 (tomelukast), prostaglandin A2, prostaglandin J2, tetradecylthioacetic acid (TTA), troglitazone (CS-045), and WY-14643 (pirinixic acid).

The preferred FXR agonists are selected from the group consisting of chenodeoxyxholic acid (CDCA), 6ECDCA, tauro-6ECDCA, 6ECDCA-NO, 6EUDCA, 6EUDCA-NO, GW4064, TR12996, TR8996, LN352, LN6733, LN6734, fexaramine and guggulsterone.

### DETAILED DESCRIPTION OF THE INVENTION

### DEFINITIONS

The following definitions are set forth to illustrate and define the meaning and scope of the various terms used to describe the invention herein.

The term “PPAR” refers to a peroxisome proliferation activated receptor. PPARs are nuclear receptors that are ligand-activated transcription factors that regulate cellular and physiological metabolism. More specifically, these receptors control lipid and glucose metabolism. PPAR alpha (PPARα) provides a target for fibrate drugs, which are used to lower triglycerides and raise HDL cholesterol. PPAR gamma (PPARγ) provides a target for glitazones (TZDs) and non-glitazone drugs in development, which are used to increase insulin sensitivity and lower blood glucose. PPAR delta (PPARδ) is thought to play a role in cholesterol transport and in raising HDL cholesterol.

A “PPARγ agonist” is a drug or chemical that works by targeting the gamma PPAR receptor isotype, leading to lower blood glucose levels in patients who suffer from pre-diabetic insulin resistance, Type II diabetes or related symptoms. Examples of PPARγ agonists are selective PPARγ agonists or modulators, dual PPARγ/α agonists, and pan PPARγ/α/δ agonists. Treatment with PPARγ agonists is known to cause dose dependent adverse side effects such as edema and weight gain in patients taking the drug. Another potential risk associated with taking PPARγ agonists is inducing cardiac failure which is often due to edema caused by excessive fluid retention. Particularly, patients who suffer from congestive heart failure (CHF) and also take insulin are at a higher risk of cardiac failure than patients who have no CHF symptoms when treated with PPARγ agonists alone. As a result, treatment with PPARγ agonists is generally not recommended for patients who are diagnosed with stage III and IV heart disease (HD).

A “dual or pan agonist” functions by targeting two or three receptor isotypes, respectively. A “dual PPARγ/α agonist” works by targeting both the alpha and gamma PPAR receptor isotypes and, as a result of this dual agonist activity, provides both blood sugar control and improved lipid management in patients who suffer from pre-diabetic insulin resistance, Type II diabetes and related conditions. A “pan PPARγ/α/δ agonist” functions by targeting the alpha, gamma and delta PPAR receptor isotypes, thereby increasing the improvement in lipid metabolism. The pan PPARγ/α/δ agonist also achieves a reduction in blood glucose and insulin, a reduction in LDL cholesterol, and an increase in HDL cholesterol. Although these dual and pan agonists provide improved treatments for patients suffering from pre-diabetic insulin resistance, Type II diabetes and related conditions, diseases and symptoms, they still cause adverse side effects to a greater or lesser extent depending on the degree of PPARγ activation.

The term “edema” refers to a condition wherein there is an abnormal accumulation of excessive fluid in body tissues, specifically in the intercellular or interstitial spaces (i.e., between the cells in the spaces that are outside of the blood vessels), and most commonly in subcutaneous tissue. Depending on severity, an excessive amount of watery fluid may be present in the tissues, which leads to observable swelling in certain parts of the body. The term includes both pitting and non-pitting edema.

The term “FXR” refers to the farnesoid X receptor, which is a bile acid-activated nuclear hormone receptor that plays a role in regulating bile acid homeostasis and cholesterol metabolism. FXR binds bile acids and regulates the rate of cholesterol degradation, bile acid biosynthesis and enterohepatic bile flow. As such, FXR is involved in the regulation of cholesterol by monitoring levels of bile acids, which are produced from cholesterol and secreted by the liver. FXR is also now known to play a role in regulating hepatic fibrogenesis that leads to cirrhosis in a variety of chronic liver diseases.

An “FXR agonist” is a chemical or drug that functions by targeting and selectively binding the farnesoid X receptor (FXR). Treatment with an FXR agonist lowers triglyceride (TG) levels in patients who suffer from pre-diabetic insulin resistance, Type II diabetes or related conditions such as hypertriglyceridemia. Treatment with an FXR agonist should prevent, limit and/or reverse fibrosis and cirrhosis in insulin resistant or Type II diabetic patients also suffering from NAFLD or NASH with progressive liver fibrosis and/or cirrhosis. An FXR agonist may further potentiate the effect of another receptor ligand. For example, an FXR agonist of the instant invention potentiates or enhances the insulin sensitizing effects,as well as the potential antifibrotic and anti-inflammatory effects, of a PPARγ agonist.

“Insulin resistance” refers to a condition wherein a person produces enough insulin, but the body does not respond efficiently to the action of insulin. As a result, the normal response of the body to a given amount of insulin in transporting glucose into cells is diminished. This may occur because the person is overweight and has too many adipose cells, which do not respond well to insulin. In addition, cells lose some of their ability to respond to insulin as they age. Insulin resistance is also linked to high blood pressure and high levels of fat in the blood stream. Notably, insulin resistance may be present in a person for a considerable number of years before the actual onset of Type II diabetes is detected and/or diagnosed.

The term “insulin sensitizing effect” means that the treatment increases the ability of insulin to reduce glucose levels by enhancing the receptor mediated effects of insulin on glucose transport in the liver, muscle and adipose tissue.

“Pre-diabetic insulin resistance” means, for the purpose of the specification and claims, abnormally high levels of blood glucose and insulin circulating in the blood. Fasting blood glucose levels in this state range around from 100 mg/dL to 125 mg/dL. Insulin resistant individuals usually also have excess abdominal fat (more than 40 inches around the waste for men and 35 inches for women), abnormally high LDL blood cholesterol levels, low HDL cholesterol levels (e.g., below 40 mg/dL for men and below 50 mg/dL for women), high levels of triglycerides (e.g., 150 mg/dL or higher), and high blood pressure (e.g., 130/85 mmHg or higher), all conditions that put these individuals at risk for cardiovascular disease.

The term “weight gain”, as used herein, refers to an increased body mass that may be drug-induced. As such, the weight gain in a person may be the result of administering a specific drug to that person. In order to determine whether a person is overweight or obese, a body mass index (BMI) is used. The BMI is a measure used to evaluate body weight relative to height. Hence, the BMI can be used to find out whether a person is underweight, normal weight, overweight, or obese. See Body Mass Index Table of Clinical Guidelines on the Identification, Evaluation, and Treatment of Overweight and Obesity in Adults: The Evidence Report (Insulin Resistance and Pre-Diabetes, NIH Publication No. 04-4893 (May 2004).

FXR Agonists to Reduce PPARγ Agonist-Induced Adverse Side Effects

The instant invention provides for the use of PPARγ and FXR agonists for the treatment of patients who suffer from drug-induced adverse side effects such as edema and weight gain. These patients further suffer from pre-diabetic insulin resistance, Type II diabetes or other related conditions. In one embodiment of the invention, the patients may also suffer from a symptom of a condition or disease such as obesity, non-alcoholic fatty liver disease (NAFLD), non-alcoholic steatohepatitis (NASH), hyperlipidemia, dyslipidemia, hypercholesterolemia, hypertriglyceridemia, high fasting blood glucose, fluid retention, edema, retinopathy, kidney disease, peripheral neuropathy, hypertension, atherosclerosis and heart failure. In a preferred embodiment of the invention, the patients may also suffer from a symptom of a condition or disease such as hypertension, atherosclerosis, peripheral vascular disease, and congestive heart failure.

The invention provides for methods of ameliorating such drug-induced adverse side effects as edema and weight gain by utilizing the therapeutic effect of PPARγ agonists in combination with FXR agonists. More specifically, the methods of the invention employ coadministering to a patient an FXR agonist in an amount that is sufficient to potentiate the insulin sensitizing effect, as well as potential antifibrotic and anti-inflammatory effects, of the PPARγ agonist, thereby reducing the amount of the PPARγ agonist taken by the patient such that the adverse side effects are lessened while the therapeutic insulin sensitizing and other beneficial effects are preserved. The PPARγ and FXR agonists that are used in this invention are well described in the medical and scientific literature.

FXR Agonists

FXR agonists activate the farnesoid X receptor [FXR; NR1H4]. FXR is a nuclear receptor expressed in tissues exposed to bile acids, such as liver, intestine, gallbladder and kidney. FXR alters gene transcription by binding DNA sequences composed of two inverted repeats separated by one nucleotide (IR-1) as a heterodimer with the 9-cis-retinoic acid (9-cis-RA) receptor (RXR). In liver, upon activation, FXR initiates a transcription of a cohort of genes that function to decrease the concentration of bile acids within the hepatocyte. Specifically, FXR induces the expression of target genes like the bile salt export pump (BSEP), multidrug resistance protein 3 (MDR3) and others that mediate bile salt excretion, the feedforward pathway. In addition, activation of FXR by both naturally occurring ligands (e.g., chenodeoxycholic acid, CDCA) and synthetic ligands leads to a feedback repression of the target genes CYP7A1 and CYP8B1, which encode sterol 7α-hydroxylase and sterol 12α-hydroxylase involved in bile acid synthesis from cholesterol. The FXR-dependent suppression of these genes is mediated by the transcriptional repressor short heterodimer partner-1 (SHP; NROB2), an atypical nuclear receptor that lacks a DNA-binding domain. In turn, SHP interacts with LRH-1, a known positive regulator of CYP7A1, and represses its transcriptional activity. Thus, FXR regulates bile acid and cholesterol metabolism.

A large number of known compounds having dissimilar chemical structures have demonstrated their ability to specifically bind and agonize FXR. For instance, WO00/40965, WO00/76523, WO03/015771, WO03/015777, WO03/016280, WO03/016288, WO03/030612, and WO03/043581 provide a long list of such compounds as potential candidates for FXR-activating ligands.

The growing list of known FXR-specific ligands includes chenodeoxyxholic acid (CDCA), 6ECDCA, GW4064, fexaramine, lithocholic acid (LCA), cholate (CA), ursodeoxycholate (UDCA), and deoxycholic acid (DCA) (Pellicciari et al., J. Med. Chem., 45:3569-3572, 2002). These compounds can be chemically synthesized according to well known methods or some of them can be purchased from commercial suppliers such as Sigma-Aldrich (USA), Erregierre (Italy), and Hengchanlong Pharmaceuticals (China).

Assays for Identifying FXR Ligands

Several assay systems have been established for identifying FXR ligands including those yet to be discovered. An FXR agonist is a compound that activates FXR by at least 40% above background in any of these assays. For example, a candidate compound can be tested in a cell-free ligand sensing assay to determine if the compound is an FXR-activating ligand and its efficacy. Briefly, this system utilizes the binding between FXR and an SRC1 peptide, which is one of the nuclear proteins known to be recruited to FXR upon FXR's binding to its ligand. The ligand-dependent recruitment of an SRC1 peptide to FXR is measured by fluorescence resonance energy transfer (FRET). For a detailed description of this assay system, see, e.g., Maloney et al., J. Med. Chem., 43:2971-2974, 2000; Pellicciari et al., J Med. Chem., 45:3569-3572, 2002.

Another assay system useful for testing a compound for its FXR ligand properties is a whole cell model involves a reporter gene (such as luciferase or β-galactosidase) controlled by a transcription regulatory element responsive to a ligand activated FXR. The level of reporter activity indicates a test compound's effectiveness as an FXR activating ligand. For a detailed description of such a reporter gene-based screening system, see, e.g., Goodwin et al., Mol. Cell., 6:517-526, 2000; Pellicciari et al., J. Med. Chem., 45:3569-3572, 2002.

In addition, there are established methods for the screening of a ligand specific for FXR and not for other nuclear receptors, particularly RXR. For example, WO 00/76523 describes an assay system in which the recombinant RXR is mutated by a single point substitution (RXRD322P) to eliminate the RXR ligand-binding site, such that the use of FXR-RXRD322P heterodimer permits unambiguous identification of compounds that are capable of modulating FXR activity.

PPAR Agonists

This invention is directed to patients who are taking PPARγ agonists and who are suffering from adverse side effects. When PPARγ agonists are combined with FXR agonists, the quantity of PPARγ agonist can be reduced and the potential drug-induced side effects of the PPARγ agonists are similarly reduced. Peroxisome proliferator activated receptors [PPAR] are a family of ligand activated transcription factors. They are members of the nuclear receptor gene family and there are three distinct isotypes or subtypes, PPAR alpha, gamma and delta. Under natural conditions, PPARs form heterodimers with other nuclear receptors such as RXR. Once activated, the receptors will stimulate a wide range of transcription events having central roles in storage and break down of fatty acids. Drugs with varying degrees of subtype specificity are known. Two good reviews of the family of PPAR are Willson T. M. et al., The PPARs: From Orphan Receptors to Drug Discovery published in the Journal of Medicinal Chemistry, 43(4):527-550 and Kliewer et al., Peroxisome Proliferator-Activated Receptors: From Genes to Physiology, Recent Prog Horm Res. 2001;56:239-63.

The PPARγ specific agonists are preferred for use in this invention. A number of PPARγ agonists and modulators have been described. For example, WO 99/38845 describes aryl substituted modulators, WO 98/02159 describes thiazolidinediones, and WO 01/30343 describes fatty acid derivatives that are agonists of PPARγ. Examples include but are not limited to, azelaoyl PAF, 2-bromohexadecanoic acid, ciglitizone, clofibrate,15-deoxy-δ¹²,¹⁴prostaglandin J₂, Fmoc-Leu-OH, GW1929, GW7647, 8(S)-hydroxy-(5Z,9E,11Z,14Z)-eicosatetraenoic acid (8(S)-HETE), leukotriene B₄, LY-171,883 (tomelukast), prostaglandin A₂, prostaglandin J₂, tetradecylthioacetic acid (TTA), troglitazone (CS-045), and WY-14643 (pirinixic acid). Variations of these PPARγ agonists are well within the scope of invention.

PPARα agonists are also known. These include fatty acids, fibrates and plasticizers. Specific assays and compounds are described in WO 97/36579. Natural ligands include palmitic acid, linoleic acid and 8(S)-HETE. Synthetic ligands include clofibrate and clofibric acid, fenofibrate and bezafibrate.

PPARδ specific agonists are also known. WO 97/28149 describes a variety of multi-ringed aromatic compounds. Willson et al., Supra describes specific agonists, both natural and synthetic compounds on page 540.

The known PPAR agonists have varying degrees of specificity to the three subtypes. Thiazolidinediones are an example of this. See U.S. Pat. No. 6,200,998. Akyl substituted aryls with dual agonist activity for PPARα/γ are described in EP 0888317. Examples include, but are not limited to, muraglitazar, galida tesaglitazar, naveglitazar-(LY8 18) and LY929.

Non-specific subtype PPAR agonists utilized herein are termed pan PPARγ/α/δ agonists. Examples include, but are not limited are, GSK's 677954 and PLX204.

Assays for Identifying PPARγ Agonists

PPAR agonists are routinely identified by a number of well known assays. A PPAR agonist is a compound that activates the PPAR by at least 10% above background in any of these assays. The subtypes of PPAR are interchangeable in these assays. For example, the PPARγ gene transcription activation assay is based on transient transfection into human HEK293 cells of two plasmids encoding a chimeric test protein and a reporter protein, respectively. The chimeric test protein is a fusion of the DNA binding domain (DBD) from the yeast GAL4 transcription factor to the ligand binding domain (LBD) of the human PPARγ protein. The PPARγ LBD has in addition to the ligand binding pocket also the native activation domain, allowing the fusion protein to function as a PPARγ ligand dependent transcription factor. The GAL4 DBD will force the fusion protein to bind only to Gal4 enhancers (of which none exist in HEK293 cells). The reporter plasmid contains a Gal4 enhancer driving the expression of the firefly luciferase protein. After transfection, HEK293 cells express the GAL4-DBD-PPARγ-LBD fusion protein. The fusion protein will in turn bind to the Gal4 enhancer controlling the luciferase, expression, and do nothing in the absence of ligand. Upon addition to the cells of a PPARγ ligand, luciferase protein will be produced in amounts corresponding to the activation of the PPARγ protein. The amount of luciferase protein is measured by light emission after addition of the appropriate substrate.

For this assay, HEK293 cells are grown in DMEM+10% FCS, 1% PS. After DNA transfection, cells are allowed to express protein for 48 hours followed by addition of compound. Human PPARγ is obtained by PCR amplification using cDNA templates from liver, intestine and adipose tissue. Amplified cDNAs are cloned into plasmids and preferably sequenced. The LBD is generated by PCR and fused to GAL4-DBD by subcloning fragments in frame into a vector (e.g., pM1 vector), thereby generating the plasmid (PM1. PPARγ.LBD). The reporter is constructed by inserting an oligonucleotide encoding five repeats of the Gal4 recognition sequence into a pGL2 vector (Promega). All compounds are dissolved in DMSO and diluted 1:1000 upon addition to the cells. Cells are treated with compound (1:1000 in 200 μl growth medium) for 24 h followed by luciferase assay. The luciferase assay is performed using the LucLite kit according to the manufacturers instructions (Packard Instruments). Light emission is quantified by counting SPC mode on a Packard Instruments top-counter. For a detailed description of this assay see U.S. Pat. No. 6,723,731. Variations of this assay are widely discussed in the scientific literature and are within the scope of this invention.

Patients Taking PPAR Agonists

The family of PPARs play a key role in lipid and fatty acid metabolism. Agonists of the three subtypes have been determined to have therapeutic benefit for persons suffering from a variety of endocrine and cardiovascular diseases. Specific diseases treatable with PPAR agonists include diabetes Type II diabetes, insulin resistance, obesity, non-alcoholic fatty liver disease (NAFLD), non-alcoholic steatohepatitis (NASH), hyperlipidemia, dyslipidemia, hypercholesterolemia, hypertriglyceridemia, high fasting blood glucose, fluid retention, retinopathy, kidney disease, peripheral neuropathy, hypertension, atherosclerosis peripheral vascular disease, and congestive heart failure.

The benefits of PPAR agonists are typically measured by improvements in the underlying disease or condition being treated. Those trained in treating the various diseases and conditions know what diagnostic criteria are appropriate to measure. This invention provides for circumstances where the FXR agonist is capable of potentiating the PPAR agonist's insulin sensitizing effect by some set percentage, (e.g., 10-30%). This is typically measured by blood glucose (both fasting and non-fasting) and HbA1C.

Patients who would benefit from PPARγ agonist therapy to treat their Type II diabetes are sometimes not able to take, or continue to take, PPARγ agonists in therapeutic doses due to their high risk for developing heart failure. More specifically, the possibility of PPARγ agonist induced edema arising in pre-diabetic or diabetic patients with heart disease is cause for concern; the presence of edema can be a sign of congestive heart failure. Pre-diabetic and diabetic patients with moderate to severe heart disease (class III or class IV cardiac functional status) are at an even higher risk.

In addition, patients with moderate to severe heart disease (class III or class IV) are often prevented from using PPARγ agonists due to increased risk factors. Hence, it is a further object of the invention to provide patients who suffer from moderate to severe heart disease (class III or class IV) with a combination therapy including TZDs and FXR agonists as described herein. This allows patients diagnosed with pre-diabetic insulin resistance or Type II diabetes with moderate to severe heart disease to benefit from PPARγ agonist treatment which would otherwise not be available to them.

Among the PPARγ agonists that have been tested in clinical trials are troglitazone, rosiglitazone and pioglitazone. These drugs are insulin sensitizers that belong to the thiazolidinedione (TZD) class of PPARγ full agonists. Rosiglitazone and pioglitazone are currently approved for the treatment of Type II diabetes and act primarily by decreasing insulin resistance. Rosiglitazone works to lower the resistance to insulin in fat-, liver- and muscle cells and also by stopping abnormalities and dysfunctions in beta-cells (see U.S. Pat. No. 5,002,953). In addition to decreasing blood sugar levels in patients, rosiglitazone also lowers triglyceride and insulin levels. Pioglitazone improves sensitivity to insulin in muscle and adipose tissue and inhibits hepatic gluconeogenesis. It further improves glycemic control while reducing circulating insulin levels. Both rosiglitazone and pioglitazone are oral glucose-lowering drugs. They are currently being tested for their anti-fibrotic and anti-inflammatory effects in the treatment of non-alcoholic steatohepatitis (NASH), atherosclerosis and other conditions and diseases.

Patients may also use TZD combination therapy (as an alternative to injected insulin) if their blood glucose levels remain too high, or if they are unable to take metformin and sulphonylurea together as a combination therapy. TZDs are effective at reducing blood glucose when added to oral monotherapy of either metformin or sulphonylurea for patients who have inadequate control of blood glucose when taking these agents alone. The combination of TZD plus metformin is preferred to TZD plus sulphonylurea, particularly for obese patients. TZD plus sulphonylurea is given to patients who show intolerance to metformin or for whom metformin is contraindicated. Because of side effects, rosiglitazone is used cautiously or is sometimes not recommended in patients with cardiac failure, hepatic impairment and severe renal insufficiency. Additional combination therapy with FXR agonists is a part of this invention.

Weight Gain

Weight gain is a primary adverse side effect. From clinical experience with diabetic patients, PPARγ agonists are associated with weight gain. Part of the weight gain may be caused by the edema-inducing effect of the agonists, but PPARγ agonists also induce adipogenesis. PPARγ agonists act preferentially on subcutaneous adipocytes which in comparison to intra-abdominal adipocytes express higher levels of PPARγ. The long-term metabolic consequences of the increased fat accumulation accompanying treatment with PPARγ agonists are not entirely known. Intra-abdominal body fat accumulation is one of several hallmarks that are typical for the metabolic syndrome and, as such, an independent risk factor of Type II diabetes (Phillip J. Larsen (September 2003) Diabetes). The combination therapy of PPARγ and FXR agonists, as contemplated by the instant invention, significantly reduces adverse side effects like edema and weight gain that are common in patients treated with PPARγ agonists alone.

From a clinical perspective, weight gain would mean an increase in body weight of at least 5% of the patients total body weight over a period of 3 to 6 months.

A considerable number of patients on PPARγ agonist therapy develop weight gain. For example, diabetic patients who take rosiglitazone at about 4-8 mg per day show a mean weight gain of about 1.9 to about 2.9 kg. A more dramatic weight gain is experienced in patients who take insulin (about 70 units per day) in addition to 4-8 mg of rosiglitazone. These patients gain on the average between about 4 to about 6 kg after six months of treatment. Similar increases in weight gain are seen in patients treated with pioglitazone, either as monotherapy or in combination with other hypoglycemic therapies (the duration of treatment may differ).

The weight gain associated with the use of TZDs is likely due to several interacting factors. Generally, improvement in glycemic control with decreased glycosuria and caloric retention may result in increased weight. The weight gain may be associated with increased subcutaneous adipose tissue and concomitant decrease in visceral fat. This change in fat distribution may in part explain the improvement in glycemic control despite the overall increase in body fat. Fluid retention such as edema, is another potential cause of increased body weight. TZDs, administered alone or in combination with other drugs (e.g., metformin, sulfonylurea, insulin) are often accompanied by an increase in plasma volume. In addition, both rosiglitazone and pioglitazone are associated with decreased levels of hemoglobin and hematocrit. These changes in weight gain and blood profile are typical for TZDs (Nesto et al., supra).

Edema

Another common side effect of treatment with PPAR agonists is edema. Edema is defined above. Edema is common in the legs, ankles, feet, hands, abdomen, around the eyes and in the lungs of people with heart failure. When edema occurs in the feet and legs it is referred to as peripheral edema. The swelling is the result of the accumulation of excess fluid under the skin. The body's organs also have interstitial spaces where fluid can accumulate. For example, an accumulation of fluid in the interstitial air spaces (alveoli) in the lungs occurs in heart failure and is called pulmonary edema. In addition, excess fluid sometimes collects in what is called the third space, which includes cavities in the abdomen (abdominal or peritoneal cavity) or in the chest (lung or pleural cavity). The term anasarca refers to the severe, widespread accumulation of fluid in the various tissues and cavities of the body.

This invention relates to PPARγ agonist-induced edema which means, for the purpose of the specification and claims, a condition wherein there is an abnormal accumulation of fluid in the body tissue(s) of a subject as a result of administering a specific drug to that subject. For example, a PPARγ agonist administered to a patient may induce a slight to severe form of edema. As such, the drug may cause peripheral edema, wherein excess fluid accumulates in the interstitial spaces under the skin leading to swelling. Alternatively, the drug may cause pulmonary edema, wherein excess fluid accumulates in the alveoli of the lungs. If a particular drug is likely to cause edema, the edema may be treated by coadministering a second drug to the patient to lessen the edema. For example, an FXR agonist may be coadministered to the patient to reduce the edema caused by the PPAR agonist.

The edema may be either pitting or non-pitting edema. Pitting edema means that when pressure is applied to an area of the skin, for example, the skin of a swollen leg, by depressing the skin with a finger, it causes an indentation in the skin that persists for some time after the release of the pressure. In fact, any form of pressure can induce the pitting of this edema. Pitting edema is caused by either systemic diseases, that is, diseases that affect the various organ systems of the body, or by local conditions involving just the affected extremities. The most common systemic diseases that are associated with edema involve the heart, liver, and kidneys. In these diseases, edema occurs primarily because of the body's retention of too much salt. The excess salt holds excess water in the interstitial tissue spaces, where the retained surplus of fluid is recognized as edema. Non-pitting edema, which usually affects the legs or arms, is defined by edema where pressure that is applied to the skin does not result in a persistent indentation. Non-pitting edema can occur in certain disorders of the lymphatic system such as lymphedema, which is a disturbance of the lymphatic circulation that may occur after a radical mastectomy, or congenital lymphedema. Another cause of non-pitting edema of the legs is called pretibial myxedema, which is a swelling over the shins that occurs in some patients with hypothyroidism (underactive thyroid gland). Non-pitting edema of the legs is difficult to treat. Diuretic medications are generally not effective, although elevation of the legs periodically during the day and compressive devices may reduce the swelling.

When used as monotherapy, the incidence of edema ranges from about 3 percent to about 5 percent for each of the presently approved TZDs. When the drugs are used in combination with other glucose-lowering agents, the incidence of edema is greater. Edema is most common in patients that are treated with a combination of TZDs and insulin. For example, rosiglitazone at 4 or 8 mg/day used in combination with insulin is associated respectively with a 13.1 percent to about 16.2 percent incidence of edema, compared with 4.7 percent in those taking insulin alone (Raskin et al. (2001) Diabetes Care 24:1226-1232). Similarly, pioglitzaone at 15 or 30 mg/day used in combination with insulin is associated with a combined 15.3 percent incidence of edema, compared to 7.0 percent in those taking insulin alone (Rubin et al. (1999) Diabetes 48 (Suppl. 1):A110). Thus, the incidence of edema is highest when either of the TZDs is combined with insulin.

Hemodilution is a test that can be used to assess if edema is present in a patient. Edema is a form of fluid retention, wherein there is an abnormal accumulation of fluid in the body tissue(s) of a patient. Edema is typically determined during a physical examination that includes blood work (e.g., testing hematocrit and hemoglobin levels) and hemodilution (i.e., decreased levels of hematocrit and/or hemoglobin). Edema often presents with symptoms such as lung congestion and ankle swelling.

Treatment with a single agent like a PPARγ agonist often leads to decreased levels of hemoglobin and hematocrit due to increased plasma volume. Increased plasma volume is a result of edema or fluid retention. Thus, measuring levels of hemoglobin and hematocrit provides an assessment of the severity of edema in a patient. A fall of hemoglobin and/or hematocrit of about 2 percent to about 4 percent is statistically significant and shows that edema is present (Nesto et al., supra). In comparison, the restoration of or increase towards normal levels of hemoglobin and/or hematocrit indicates a clinically significant attenuation of edema due to PPARγ agonist treatment.

The efficacy of treatment with a combination of PPARγ and FXR agonists can be determined by a reduction in projected incidence and amount of edema which can be measured via hemodilution monitoring of patients (infra). The same patient may also experience less than anticipated or no weight gain,which can be measured by carefully monitoring a patient's body weight and by calculating the patient's BMI (supra) before and after the treatment with PPARγ and FXR agonists.

Formulation

PPARγ and FXR agonists are formulated as pharmaceuticals to be used in the methods of the invention. Any composition or compound that can stimulate a biological response associated with the binding of a ligand analogue (i.e., an agonist or modulator) to PPARγ and FXR can be used as a pharmaceutical in the invention. General details on techniques for formulation and administration are well described in the scientific literature (see “Remington's Pharmaceutical Sciences”, Maack Publishing Co, Easton Pa.). PPARγ and FXR agonist pharmaceutical formulations can be prepared according to any method known in the art for the manufacture of pharmaceuticals. The PPARγ and FXR agonists used in the methods of the invention can be formulated for administration in any conventionally acceptable way including via intravenous injection, IM, IP, orally, topically, vaginally or rectally. Oral administration is preferred. Illustrative examples are set forth below.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, capsules, liquids, lozenges, gels, syrups, slurries, suspensions, etc., suitable for ingestion by the patient. Pharmaceutical preparations for oral use can be obtained through combination of PPARγ and FXR agonist compounds with a solid excipient, optionally grinding a resulting mixture, and processing the mixture of granules, after adding suitable additional compounds, if desired, to obtain tablets or pills. Suitable solid excipients are carbohydrate or protein fillers which include, but are not limited to, sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Pharmaceutical preparations of the invention that can also be used orally are, for example, push-fit capsules made of gelatin, as well as soft, sealed capsules made of gelatin and a coating such as glycerol or sorbitol. Push-fit capsules can contain PPARγ and FXR agonists mixed with a filler or binders such as lactose or starches, lubricants such as talc or magnesium stearate, and, optionally, stabilizers. In soft capsules, the PPARγ and FXR agonist compounds may be dissolved or suspended in suitable liquids, such as fatty oils, liquid paraffin, or liquid polyethylene glycol with or without stabilizers.

Aqueous suspensions of the invention contain a PPARγ or FXR agonist in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a PPARγ or FXR agonist in a vegetable oil, such as arachis oil, olive oil, sesame oil or coconut oil, or in a mineral oil such as liquid paraffin. The oil suspensions can contain a thickening agent, such as beeswax, hard paraffin or cetyl alcohol. Sweetening agents can be added to provide a palatable oral preparation. These formulations can be preserved by the addition of an antioxidant such as ascorbic acid.

Dispersible powders and granules of the invention suitable for preparation of an aqueous suspension by the addition of water can be formulated from a PPARγ or FXR agonist in admixture with a dispersing, suspending and/or wetting agent, and one or more preservatives. Suitable dispersing or wetting agents and suspending agents are exemplified by those disclosed above. Additional excipients, for example sweetening, flavoring and coloring agents, can also be present.

The pharmaceutical formulations of the invention can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening and flavoring agents. Syrups and elixirs can be formulated with sweetening agents, such as glycerol, sorbitol or sucrose. Such formulations can also contain a demulcent, a preservative, a flavoring or a coloring agent.

When the drugs are delivered by intravenous injection, the PPARγ and FXR agonist pharmaceutical formulations of the invention can be in the form of a sterile injectable preparation, such as a sterile injectable aqueous or oleaginous suspension. This suspension can be formulated according to the known art using those suitable dispersing or wetting agents and suspending agents which have been mentioned above. The sterile injectable preparation can also be a sterile injectable solution or suspension in a nontoxic parenterally-acceptable diluent or solvent. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

Administration and Dosing Regimen of PPARγ Agonists and FXR Agonists

The PPARγ and FXR agonists used in the methods of the invention can be administered in any conventionally acceptable way including via intravenous injection, IM, IP, orally, topically, vaginally or rectally. Oral administration is preferred. Administration will vary with the pharmacokinetics and other properties of the drugs and the patients' condition of health. General guidelines are presented below.

The methods of the invention reduce adverse side effects in patients who suffer from insulin resistance, Type II diabetes, and/or related conditions. The amount of PPARγ agonist in combination with FXR agonist that is adequate to accomplish this is considered the therapeutically effective dose. Precise dose schedules cannot be stated. The dosage schedule and amounts effective for this use, i.e., the “dosing regimen,” will depend upon a variety of factors, including the stage of the disease or condition, the severity of the disease or condition, the severity of the adverse side effects, the general state of the patient's health, the patient's physical status, age and the like. In calculating the dosage regimen for a patient, the mode of administration is also taken into consideration. The dosage regimen must also take into consideration the pharmacokinetics, i.e., the agonist rate of absorption, bioavailability, metabolism, clearance, and the like (see, For example, Wagstaff A J, Goa K L. “Rosiglitazone: a review of its use in the management of type 2 diabetes mellitus”, Drugs. 2002;62(12):1805-37.

The state of the art allows the clinician to determine the dosage regimen for each individual patient, PPARγ agonists in combination with FXR agonists, and disease or condition treated. As an illustrative example, the guidelines provided below for PPARγ and FXR agonists can be used as guidance to determine the dosage regimen, i.e., dose schedule and dosage levels, of any PPARγ and FXR agonist administered when practicing the methods of the invention. As a general guideline, it is expected that the daily dose of PPARγ agonist of typically between 0.5mg to 100 mg/day will be reduced by between 10 percent and 90 percent and more likely between 25% and 50% by the co-administration of the FXR agonist. FXR agonists are effective in a daily dose range of between 10 mg to 400 mg. All doses are for a person of about 70 kilogram weight.

Single or multiple administrations of PPARγ agonist formulations may be administered depending on the dosage and frequency as required and tolerated by the patient who suffers from pre-diabetic insulin resistance, Type II diabetes, metabolic syndrome, non-alcoholic fatty liver disease (NAFLD) and non-alcoholic steatohepatitis (NASH), heart disease (HD) and/or related conditions. The formulations should provide a sufficient quantity of PPARγ agonist to effectively ameliorate the condition. A typical pharmaceutical formulation for oral administration of PPARγ agonist would depend on the specific agonist. For example, rosiglitazone may be administered to a patient through monotherapy (i.e., with no other diabetes medications) or in combination therapy with metformin or sulfonylurea pills.

In a preferred embodiment, rosiglitazone is currently administered to a patient daily as monotherapy or in combination with a second agent selected from the group consisting of metformin, sulfonylurea or insulin in an amount from 4 mg to 8 mg po. Similarly, pioglitazone may be administered to a patient daily as monotherapy or in combination with a second agent selected from the group consisting of metformin, sulfonylurea or insulin in an amount from 15 mg to 45 mg po(per day). With the addition of an FXR agonist that dosage is reduced by 50 percent, for example from a daily dose of rosiglitazone of 4mg po to 2 mg po.

Notably, the dosages of selective PPARγ agonists such as rosiglitazone or pioglitazone administered to a patient may vary depending on age, degree of illness, drug tolerance, and concomitant medications and conditions. The FXR agonist may be administered to the patient in combination with a PPARγ agonist in order to potentiate the insulin sensitizing effect of the PPARγ agonist (e.g., rosiglitazone, pioglitazone, etc.) and in order to reduce adverse side effects such as edema and/or weight gain. In a preferred embodiment, the invention provides for a method of reducing adverse side effects in a patient treated with a PPARγ agonist by administering an FXR agonist in a daily amount of between 0.1 mg/kg qd and about 10 mg/kg po per day. Using this dosage of FXR agonist, the daily co-administration of PPARγ agonist will be reduced by between 10 percent and 90 percent and the combination therapy will continue until the combination FXR agonist and PPARγ agonist treatment is no longer deemed beneficial or necessary.

The PPARγ and FXR agonists may be administered to a patient simultaneously or within specific time frames of one another. Preferably, the PPARγ- and FXR agonists are administered to the patient simultaneously in separate pills or tablets or in the form of a combination pill.

### EXAMPLES

The following specific examples are intended to illustrate the invention and should not be construed as limiting the scope of the claims.

I. FXR Ligands Regulate PPARγ Expression in Hepatic Stellate Cells (HSCs)

Hepatic fibrosis is a scarring process of the liver that includes both increased and altered deposition of extracellular matrix (ECM) components. In chronic liver disease, hepatic stellate cells (HSCs) undergo a process of trans-differentiation from a resting, fat-storing phenotype towards a myofibroblast-like phenotype characterized by expression of fibroblastic cell markers such as α1 (I) collagen and α-smooth muscle actin (α-SMA). The members of the nuclear receptor (NR) superfamily (e.g., PPARs) are believed to exert counteregulatory effects acting as a braking signal to prevent HSC trans-differentiation.

FXR ligands increase PPARα mRNA expression in human hepatocytes (see Pineda Torra et al. (2003) Mol. Endocrinol. 17:259-72.). However, whether or not FXR interacts with PPARγ, has so far been unknown. In order to investigate if FXR interacts with PPARγ, in vitro studies were performed on primary cultures of rat HSCs and HSC-T6 (i.e., a rat immortalised HSCs line). Primary rat HSCs were isolated from control and cirrhotic rats according to techniques known in the art. The HSCs were more than 90% viable as assessed by trypan blue exclusion and greater than 95% pure. Cells were cultured at 37° C. in an atmosphere of 5% CO₂ in Dulbecco's modified minimal essential medium (Gibco BRL Life Technologies, Rockville, Md.) containing 10% fetal calf serum (FCS), 2 mM L-glutamine, and 5,000 IU/ml penicillin/5,000 g/ml streptomycin. In order to investigate the expression of FXR and PPARα, PPAR-β and PPAR-γ in HSCs, and the effect of FXR and PPARγ ligands on HSC activation, a primary culture of rat HSCs (day 0 and day 7) and 24 hours starved HSC-T6 cells were incubated for 18 hours with medium alone or increasing concentrations of 6-ECDCA, i.e., a semi-synthetic derivative of CDCA (0.1-10 EM); GW4064, i.e., a non-steroidal FXR ligand (0.01-1 μM); and rosiglitazone, i.e., a PPARγ ligand (0,1-10 μM). The mRNA expression for FXR, PPARs, α1(I) collagen, SHP, TIMP-1, TIMP-2, MMP-2 and TGFβ1 was investigated by quantitative (q)RT-PCR (see Fiorucci et al. (2005) J. Pharmacol. Exp. Ther. 315(1):58-68). HSCs were incubated with or without 6-ECDCA, 1 μM, for 24 hours at 37° C. in DMEM. Cell lysates were prepared by solubilization of cells in sample buffer (62.5 mM Tris-HCl, pH 6.8, 10% glycerol, 2% SDS, 0.015% bromophenol blue) and separated by polyacrylamide gel electrophoresis (PAGE). The proteins were then transferred to nitrocellulose membranes (Biorad, Hercules, Calif.) and probed with primary antibodies to FXR and PPARγ (Santa Cruz Biotechnology, Santa Cruz, Calif.). The anti-immunoglobulin G horseradish peroxidase conjugate (Biorad, Hercules, Calif.) was used as the secondary antibody and specific protein bands were visualized using enhanced chemiluminescence (ECL) (Amersham Biotechnology Pharmacia, Piscataway, N.J.) following the manufacturer's suggested protocol.

PPARγ expression normally decreases during HSC trans-differentiation. Thus, to investigate whether FXR ligands could reverse this pattern, primary cultures of HSCs were grown in plastic dishes for seven days with or without the synthetic FXR ligand, 6-ECDCA. Notably, culturing the cells with 1 μM 6-ECDCA caused a 40 fold increase in PPARγ mRNA and protein. Similarly, both natural and synthetic FXR ligands, CDCA and GW4064 respectively, prevented the downregulation of PPAR-γ caused by HSCs activation. To further investigate whether ligands of FXR and PPARγ might cooperate in repressing α1 (1) collagen gene expression, HSC-T6 were exposed to effective concentrations of the two ligands. While 0.1 μM 6-ECDCA and 1 μM rosiglitazone individually decreased α1 (1) collagen and α-smooth muscle actin (α-SMA) mRNA by 30-40%, the combination of the two led to a significant increase in this effect, resulting in a 3-fold induction of PPARγ and about 80% reduction of α1 (1) collagen and α-SMA mRNA (n=4; P<0.05 versus 6-ECDCA or rosiglitazione alone). Similar to rosiglitazone, co-incubation of HSCs with 6-ECDCA in combination with pioglitazone and 15-deoxy-Δ12,14-prostaglandin J2 (PGJ2) (i.e., the natural ligand of PPARγ), resulted in a significant additive effect in repressing TGFβ1-regulated genes (n=5; P<0.05 versus pioglitazone or PGJ2 alone).

Thus, it is shown herein that natural and synthetic FXR ligands induce PPARγ expression in HSCs and further, that the FXR ligand protects against PPARγ downregulation caused by liver diseases and enhances the activity of PPARγ ligands. These findings provide the first molecular evidence for cross-talk between FXR and PPARγ.

II. FXR Ligands Regulate PPARγ Expression In Vivo in Liver Fibrosis

In order to further investigate whether in vivo administration of FXR ligands would modulate the expression of PPARγ, three different models of liver fibrosis were used, i.e., porcine serum administration, bile duct ligation (BDL), and CCL₄ intoxication. Male Wistar rats (200-250 g) were obtained from Charles River Breeding Laboratories (Monza, Italy) and maintained on standard laboratory rat chow on a 12 hour light/dark cycle.

In the first model, liver fibrosis was induced by repeated intraperitoneal (i.p.) administrations of 0.5 ml of porcine serum twice a week for 8 weeks (see Fiorucci et al. (2004) Gastroenterology 14:1444-1356). To investigate whether 6-ECDCA was effective in regulating PPARγ expression, porcine serum-administered rats (6-8 each group) were randomized to receive 1 and 3 mg/kg 6-ECDCA via gavage 5 times a week. Control rats were administered 3% carboxy-methyl cellulose (CMC) by gavage. At the end of the study, rats were sacrificed under anaesthesia with sodium pentobarbital (50 mg/kg, i.p.) and terminally bled via cardiac puncture. The liver was removed for examination and blood samples were taken.

In the second model, hepatic fibrosis was induced by bile duct ligation (BDL) of 8-9 weeks old male Wistar rats (see Fiorucci et al. (2004) Gastroenterology 14:1444-1356 and Fiorucci et al. (2004) Hepatology 39:365-75). Sham operated rats (n=6) received the same laparoscopic procedure, except that the bile duct was manipulated, but not ligated and sectioned. A total of 24 animals were operated. Two weeks after surgery surviving rats were randomized to receive placebo, i.e., 3% CMC (6 rats) or 6-ECDCA, 3 mg/kg (8 rats) by gavage. Animals were then treated for 14 days.

In the third model, liver fibrosis was induced in rats by i.p. injection of CCl₄, 100 μl/100 g body weight in an equal volume of paraffin oil two times a week for 4 weeks. Control rats were injected i.p. with 100 μl/100 g body weight of paraffin oil alone. Rats (6 per group) were then treated by oral administration of 3 mg/kg 6-ECDCA in CMC five times a week or 3% CMC alone (control) for 8 weeks.

When rats were treated with the FXR ligand it resulted in a robust induction of PPARγ expression in all three models. While treatment with 3 mg/kg 6-ECDCA increased FXR and SHP mRNA by 1.8 to 4 fold respectively, the FXR ligand increased PPARγ mRNA expression by 3 to 5 fold.

In another set of experiments, it was investigated whether 6-ECDCA interacts with rosiglitazone on liver fibrosis induced by porcine serum administration. Groups and duration of treatment are described in Table 1 below. Animals were followed for 8 weeks.

TABLE 1
Effect of FXR and PPARγ Ligands on Liver Fibrosis Induced By 8-week
Administration of Porcine Serum to Rats (PS).

| | Number | | Liver/b.w. | ALT | ALP | Bilirubin |
|---|---|---|---|---|---|---|
| Treatment | of rats | Final b.w. (g) | (%) | (U/L) | (U/dl) | (mg/dl) |
| Control | 6 | 376.0 ± 10.5 | 2.60 ± 0.5 | 34.3 ± 4.4 | 22.5 ± 3.6 | 0.1 ± 0.01 |
| PS (8 wks) | 10 | 354.3 ± 12.3 | 4.1 ± 0.5* | 46.4 ± 5.4 | 29.0 ± 5.2 | 0.3 ± 0.01 |
| PS + 6-ECDCA (1 mg/kg) | 10 | 357.3 ± 9.8 | 3.8 ± 0.3 | 47.4 ± 2.4 | 26.6 ± 2.5 | 0.3 ± 0.01 |
| PS + 6-ECDCA (3 mg/kg) | 10 | 352.4 ± 10.3 | 2.9 ± 0.2 | 41.9 ± 5.3 | 21.8 ± 5.2 | 0.2 ± 0.01 |
| PS + Rosiglitazone (1 mg/kg) | 10 | 346.9 ± 11.5 | 4.0 ± 0.3 | 47.6 + 4.2 | 23.4 ± 3.7 | 0.3 ± 0.06 |
| PS + Rosiglitazone (3 mg/kg) | 10 | 358.3 ± 11.8 | 2.81 ± 0.4 | 40.7 ± 5.2 | 25.6 ± 3.1 | 0.3 ± 0.07 |
| PS + 6-ECDCA (1 mg/kg) + | 10 | 361.4 ± 8.9 | 3.1 ± 0.5 | 40.4 ± 4.2 | 28.9 ± 2.7 | 0.1 ± 0.02 |
| rosiglitazione (1 mg/kg) | | | | | | |
| PS + 6-ECDCA (3 mg/kg) + | 10 | 369.6 ± 10.8 | 2.7 ± 0.4** | 37.5 ± 3.3 | 22 ± 4.2 | 0.2 ± 0.01 |
| rosiglitazione (3 mg/kg) | | | | | | |

Rats were treated twice a week for 8 weeks with repeated intraperitoneal injections of either saline or porcine serum along with oral administrations of FXR and PPARγ ligands. The body weight was measured immediately before sacrificing. After removal of the liver, it was weighed and the ratio to whole-body weight was then calculated.
Data are mean ± SE of indicated number of rats.
*P < 0.05 versus control rats
**P < 0.05 versus rats administered porcine serum alone

For histological examination, portions of the right and left liver lobes (10-15 mg/each) from each animal were fixed in 10% formalin, embedded in paraffin, sectioned, and stained with Sirius red (see Fiorucci et al., supra; and Lopez-De Leon et al. (1985) J. Histochem. Cytochem. 33: 737-743). Collagen surface density was quantified using a computerized image analysis system (Image Acquisition System Ver. 005, Delta Sistemi, Rome, Italy) (see Fiorucci et al., supra). Hepatic and urinary content of hydroxyproline were determined by HPLC (LC Varian Prostar HPLC, Varian, Rome, Italy) as described in Fiorucci et al. (supra). The ANOVA followed by Dunnett or Bonferroni correction for multiple comparison were applied when appropriate during statistical analysis. EC₅₀ were calculated using Prism III (Graphpad Software. Inc., San Diego, Calif.).

Histological evaluation of liver specimens obtained from rats administered with porcine serum for 8 weeks showed extensive perilobular fibrosis resulting in a 10-fold increase of the surface area of hepatic collagen in comparison with control rats. The number of A-SMA positive HSCs in the fibrous septa increased significantly in cirrhotic rats compared to control rats. Administration of 1 and 3 mg/kg of 6-ECDCA and rosiglitazone, respectively, did not affect liver function as measured by plasma ALT, alkaline phosphatase and bilirubin (P>0.05 versus control and porcine serum-treated rats). However, both drugs effectively protected rats against development of liver fibrosis at the dose of 3 mg/kg (see Table 1). While expression of selected pro-fibrogenetic markers was reduced in animals treated with 1 mg/kg 6-ECDCA (α-SMA, fibronectin, TGFP1), this dose slightly reduced the histological score and the liver hydroxyproline content in comparison to animals treated with porcine serum alone. No significant effects were observed in any biochemical or molecular marker of liver fibrosis in animals treated with 1 mg/kg rosiglitazione. In contrast, administration of rats with 3 mg/kg 6-ECDCA or rosiglitazone decreased the area of liver parenchyma occupied by fibrotic tissue and hepatic levels of hydroxyproline as well as expression of α-SMA, α1 (I) collagen, fibronectin, TGFβ1, TIMP-1 and TIMP-2 mRNAs by 50-60% in comparison with rats administered porcine serum alone (n=8 to 12; P<0.01 versus porcine serum alone). Co-administration of 1 mg/kg 6-ECDCA together with 1 mg/kg rosiglitazone resulted in higher anti-fibrotic activity with respect to that observed with either of the two drugs alone and reduced the extent of liver fibrosis as measured by morphometric analysis by 50-60% in comparison with porcine serum alone (n=8 to 12; P<0.01 versus porcine serum alone). Similarly, co-administration of 6-ECDCA and rosiglitazone at the dose 3 mg/kg each resulted in a significant potentiation of the antifibrotic effect exerted separately by the two drugs. Indeed, this combination ameliorated the histologic score, reduced the liver hydroxyproline content and decreased the expression of α-SMA, α1 (I) collagen, fibronectin, TGFβ1, TIMP-1 and TIMP-2 mRNAs by about 90% (P<0.05 versus 6-ECDCA and rosiglitazone). The beneficial effect observed in rats treated with the combination of FXR and PPARγ ligands correlated with a significant induction of PPARγ mRNA expression in the liver. Thus, while administering rats with 1 mg/kg 6-ECDCA or 1 and 3 mg/kg roglitazone alone increased PPARγ mRNA by 1-2 fold, administration of 3 mg/kg 6-ECDCA resulted in 3-4 fold induction. Further, similarly to rosiglitazone, 6-ECDCA (3 mg/kg) increased the hepatic expression of UCP-2, a PPARγ regulated gene, by 2-4 fold (P<0.05 versus control and porcine serum alone).

Both in vivo and in vitro data have shown that FXR ligands can reverse downregulation of PPARγ. It was demonstrated that while treatment of rats with 1 mg/kg 6-ECDCA and rosiglitazone alone, i.e., sub-maximally effective doses of these agents, caused a 20-30% reduction of markers of hepatic fibrosis, co-administration of the two ligands at these doses reduces liver fibrosis as assessed by liver morphometry by about 60%. Furthermore, while a significant reduction in liver fibrosis was observed in rats treated with 3 mg/kg of both agents alone, administration of the combination of 6-ECDCA and rosiglitazone at 3 mg/kg for 8 weeks resulted in a 90% reduction in liver collagen content. The reduction in collagen deposition obtained by the combination of FXR and PPARγ ligands was associated with a reduction in the parenchymal area occupied by α-SMA-positive cells, suggesting a causal relationship between the decreased number of activated HSCs and the reduced accumulation of ECM components. Protection against development of liver fibrosis induced by 6-ECDCA associated with a significant induction of PPARγ and SHP gene expression.

The above experiments have demonstrated that FXR ligands regulate PPARγ gene expression and that FXR and PPARγ ligands synergize in regulating fibrogenic activities of HSCs. While it was shown here that the effect of FXR ligands are additive to the effects of PPARγ ligands in reducing liver fibrosis, these results extend to other diseases as well. Indeed, FXR ligands increase the expression of UCP-2, a PPARγ regulated gene involved in regulation of energy metabolism which suggests that FXR ligands enhance the glucose lowering effects of PPARγ ligands. The synergistic activity of FXR ligands and PPARγ ligands is believed to contribute to limit the incidence of side-effects associated with the use of PPARγ agonists. In fact, 6-15% of diabetic patients taking rosiglitazone or pioglitazone are known to develop a diuretic resistant edema. Since the incidence of side effects caused by these two drugs is dose-dependent, a combination of FXR and PPARγ ligands would contribute to limit the dose of PPARγ ligand, reducing the burden of side effects associated with their use.

III. Potentiation of a PPARγ Agonist with an FXR Agonist in a Human Patient

A female non-insulin dependent Type II diabetic obese patient aged 48 has been taking rosiglitazone 4 mg daily by mouth for 6 weeks, after having first been started 4 weeks before at a higher dose of 8 mg daily that she could not tolerate. Since initiating TZD therapy, she has gained 6 kg, shows signs of increasing peripheral edema and has started complaining of shortness of breath and difficulty sleeping, with suboptimal glycemic control. The patient is started on a combination of 6ECDCA (150 mg) and rosiglitizone (2 mg) daily by mouth. At a two week follow up examination, she has lost 3 kg, demonstrates markedly reduced edema and has good glycemic control.

Various modifications and variations of the present invention will be apparent to those skilled in the art without departing from the scope and spirit of the invention. Although the invention has been described in connection with specific preferred embodiments, it should be understood that the invention as claimed should not be unduly limited to to such specific embodiments. Indeed, various modifications of the described modes for carrying out the invention which are obvious to those skilled in the art are intended to be within the scope of the claims.

What is claimed is:

1. A method of reducing adverse side effects in a human subject suffering from side effects induced by a PPARγ agonist, said method comprising:
(i) coadministering to the human subject an FXR agonist in an amount sufficient to potentiate an insulin sensitizing effect of the PPARγ agonist; and
(ii) reducing the amount of the PPARγ agonist taken by the human subject such that said side effects are lessened while the insulin sensitizing effect is preserved, wherein said side effects are selected from the group consisting of edema and weight gain.

2. The method of claim 1, wherein said FXR agonist achieves a 25% potentiation of the insulin sensitizing effect of the PPARγ agonist.

3. The method of claim 1, wherein said human subject is a patient suffering from pre-diabetic insulin resistance or Type II diabetes.

4. The method of claim 3, wherein the patient further suffers from a disease, condition, sign or symptom selected from the group consisting of insulin resistance, obesity, non-alcoholic fatty liver disease (NAFLD), non-alcoholic steatohepatitis (NASH), liver fibrosis, liver cirrhosis, portal hypertension, hyperlipidemia, dyslipidemia, hypercholesterolemia, hypertriglyceridemia, high fasting blood glucose, fluid retention, edema, retinopathy, kidney disease, peripheral neuropathy, hypertension, atherosclerosis and heart failure.

5. The method of claim 1, wherein said human subject is a patient suffering from symptoms selected from the group consisting of hypertension, atherosclerosis, peripheral vascular disease, and congestive heart failure.

6. The method of claim 1, wherein said PPARγ agonist is selected from the group consisting of rosiglitazone and pioglitazone.

7. The method of claim 6, wherein said rosiglitazone is administered alone or in combination with a second agent selected from the group consisting of metformin, sulfonylurea or insulin to a patient in an amount from about 0.5 mg to about 8 mg po qd.

8. The method of claim 6, wherein said pioglitazone is administered alone or in combination with a second agent selected from the group consisting of metformin, sulfonylurea or insulin to a patient in an amount from about 3 mg to about 45 mg po qd.

9. The method of claim 1, wherein said PPARγ agonist is selected from the group consisting of PPARγ modulators, dual PPARγ/α agonists and pan PPARγ/α/δ agonists.

10. The method of claim 9, wherein said PPARγ modulator is selected from the group consisting of MBX-102 and T131.

11. The method of claim 9, wherein said dual PPARγ/α agonist is selected
from the group consisting of muraglitazar, galida tesaglitazar, naveglitazar (LY818) and LY929.

12. The method of claim 9, wherein said pan PPARγ/α/δ agonist is
selected from the group consisting of GSK 677954 and PLX204.

13. The method of claim 1, wherein said amount of the FXR agonist is
between about 0.1 mg/kg qd and about 10 mg/kg qd.

14. The method of claim 1, wherein the reduced amount of PPARγ agonist
achieves a therapeutic effect in the human subject at a lower dose while lessening the side effects.

15. The method of claim 1, wherein the human subject is a patient
suffering from heart disease, wherein said patient is intolerant of being treated with a PPARγ agonist alone or in combination with a second agent selected from the group consisting of metformin, sulfonylurea and insulin.

16. The method of claim 1, wherein said PPARγ agonist is selected from.
the group consisting of azelaoyl PAF, 2-bromohexadecanoic acid, ciglitizone, clofibrate,15-deoxy-δ¹²,¹⁴ prostaglandin J₂, fenofibrate, Fmoc-Leu-OH, GW1929, GW7647, 8(S)-hydroxy-(5Z,9E,11Z,14Z)-eicosatetraenoic acid (8(S)-HETE), leukotriene B₄, LY-171,883 (tomelukast), prostaglandin A₂, prostaglandin J₂, tetradecylthioacetic acid (TTA), troglitazone (CS-045), and WY-14643 (pirinixic acid).

17. The method of claim 1, wherein said FXR agonist is selected from the.
group consisting of chenodeoxyxholic acid (CDCA), 6ECDCA, tauro-6ECDCA, 6ECDCA-NO, 6EUDCA, 6EUDCA-NO, GW4064, TR12996, TR8996, LN352, LN6733, LN6734, fexaramine and guggulsterone.

18. The method of claim 1, wherein said FXR agonist is 6ECDCA.

19. The method of claim 1, wherein the side effects are lessened such that.
there is a reduction in weight gain.

20. The method of claim 19, wherein the reduction in weight gain is quantified against actual levels of weight gain clinically observed in a patient being treated with a PPARγ agonist or against projected levels of weight gain in a patient over time, wherein said projected levels of weight gain are determined through clinical observation of patients treated over time with a PPARγ agonist.

21. The method of claim 19, wherein said patient is treated with a PPARγ agonist in combination with a second agent selected from the group consisting of metformin, sulfonylurea and insulin.

22. The method of claim 1, wherein the side effects are lessened such that there is a reduction in edema.

23. The method of claim 22, wherein the reduction in edema is quantified against actual levels of edema clinically observed in a patient being treated with a PPARγ agonist or against projected levels of edema in a patient over time, wherein said projected levels of edema are determined through clinical observation of patients treated over time with a PPARγ agonist.

24. The method of claim 22, wherein the reduction in edema results in a reduced risk of developing or worsening congestive heart failure in the human subject.

25. The method of claim 22, wherein the reduced risk of developing or worsening congestive heart failure in the human subject enables a patient suffering from advanced heart disease, who is unable to safely tolerate treatment with a therapeutically effective dose of a PPARγ agonist, to be treated safely and effectively with a combination of a PPARγ agonist and FXR agonist.

26. The method of claim 1, wherein said insulin sensitizing effect of the PPARγ agonist remains as potent at a lower dose as compared to a higher dose as a result of coadministration of the FXR agonist to the human subject.

27. A method of treating a patient with a combination of a PPARγ agonist and FXR agonist, said method comprising:
(i) coadministering to the patient an FXR agonist in an amount sufficient to potentiate an insulin sensitizing effect of the PPARγ agonist; and
(ii) reducing the amount of the PPARγ agonist taken by the patient such that said side effects are lessened while the insulin sensitizing effect is preserved, wherein said side effects are selected from the group consisting of edema and weight gain.

28. The method of claim 27, wherein said FXR agonist achieves a 25% potentiation of the insulin sensitizing effect of the PPARγ agonist.

29. The method of claim 27, wherein said PPARγ agonist selected from the group consisting of rosiglitazone and pioglitazone.

30. The method of claim 27, wherein said PPARγ agonist is a dual PPARγ/α agonist selected from the group consisting of muraglitazar, galida tesaglitazar, naveglitazar (LY818) and LY929.

31. The method of claim 27, wherein said PPARγ agonist is a pan PPARγ/α/δ agonist selected from the group consisting of GSK 677954 and PLX204.
